(19) United States
(12) Patent Application Publication
(10) Pub. No.: US 2026/0007335 A1
(43) Pub. Date: Jan. 8, 2026

(54) APPARATUS AND METHOD FOR NON-INVASIVE BLOOD GLUCOSE MONITORING

(71) Applicant: ELECTRONICS AND TELECOMMUNICATIONS RESEARCH INSTITUTE, Daejeon (KR)

(72) Inventors: Won Kyoung LEE, Daejeon (KR); Eon Sang KIM, Daejeon (KR); Heuk PARK, Daejeon (KR); Jun Ki LEE, Daejeon (KR); Seung Hyun CHO, Daejeon (KR)

(73) Assignee: ELECTRONICS AND TELECOMMUNICATIONS RESEARCH INSTITUTE, Daejeon (KR)

(21) Appl. No.: 19/258,459

(22) Filed: Jul. 2, 2025

(30) Foreign Application Priority Data
Jul. 2, 2024 (KR) 10-2024-0087138

(51) Int. Cl. A61B 5/145; A61B 3/00; A61B 5/00; A61B 5/0507; A61B 5/1455

(52) U.S. Cl. CPC A61B 5/14532; A61B 3/0025; A61B 5/0507; A61B 5/1455; A61B 5/7228; A61B 2560/0209

(57) ABSTRACT

The present invention relates to apparatus for non-invasive blood glucose monitoring, which includes: a signal transmitting module that transmits a terahertz wireless signal modulated into a PRBS pattern at a terahertz carrier frequency to a body part; a signal receiving module that demodulates the terahertz wireless signal reflected from the body part to measure performance parameters of the modulation pattern; and a sensing module that performs multivariate analysis using the performance parameters of the modulation pattern measured through the signal receiving module and quantifies features related to blood glucose through the multivariate analysis to measure blood glucose concentration.

### CROSS-REFERENCE TO RELATED APPLICATION

This application claims priority to and the benefit of Korean Patent Application No. 10-2024-0087138, filed on Jul. 2, 2024, the disclosure of which is incorporated herein by reference in its entirety.

### BACKGROUND

### 1. Field of the Invention

The present invention relates to an apparatus and method for non-invasive blood glucose monitoring using terahertz wireless communication technology.

### 2. Discussion of Related Art

Various technologies for non-invasive blood glucose monitoring have been researched to relieve diabetes patients from the pain of blood sampling.

Blood glucose measurement technologies are largely classified into minimally invasive and non-invasive methods depending on whether a glucose measurement sensor is inserted into human tissue.

Minimally invasive methods use small probes or implantable sensors to measure glucose concentration in interstitial fluid that is bodily fluid between tissue cells outside blood vessels, enabling the measurement of the glucose concentration generally without significant bleeding.

Non-invasive measurement methods are innovative technologies for measuring glucose concentration outside skin tissue without damaging any tissue. Examples of non-invasive measurement methods may include an extracorporeal fluid-type non-invasive measurement technology of measuring glucose concentration in bodily fluid discharged outside the body, such as tears and sweat.

In order to measure signals from human tissues using a non-invasive measurement method, physical signals need to be applied. As the applied physical signals, various types of physical signals, such as light, heat, electromagnetic waves, ultrasound, and fusion signals, are being used.

However, since existing non-invasive measurement methods do not have high resolution and accuracy, technology is needed to improve the resolution and accuracy.

### SUMMARY OF THE INVENTION

The present invention is directed to providing an apparatus and method for non-invasive blood glucose monitoring using terahertz wireless communication technology to improve resolution and accuracy.

According to an aspect of the present invention, there is provided an apparatus for non-invasive blood glucose monitoring, which includes: a signal transmitting module that transmits a terahertz wireless signal modulated into a pseudo-random bit sequence (PRBS) pattern at a terahertz carrier frequency to a body part; a signal receiving module that demodulates the terahertz wireless signal reflected from the body part to measure performance parameters of the modulation pattern; and a sensing module that performs multivariate analysis using the performance parameters of the modulation pattern measured through the signal receiving module and quantifies features related to blood glucose through the multivariate analysis to measure blood glucose concentration.

The signal transmitting module may beat two laser optical signals with different wavelengths using a photo mixer to generate a terahertz wireless signal corresponding to a wavelength difference between the two laser optical signals.

The signal transmitting module may project a femtosecond laser into a photoconductor to generate a terahertz signal.

The signal transmitting module may use an electronic-based terahertz transmitter to reduce power consumption.

The signal receiving module may remove a carrier frequency from the terahertz wireless signal reflected from the body part through a subharmonic mixer, down-convert the terahertz wireless signal into a signal at a frequency in a baseband, and then reconstruct the modulation pattern to extract performance parameters of distorted modulation pattern proportional to the blood glucose concentration of the body part from a three-dimensional eye diagram.

The signal receiving module may extract a histogram representing a statistical count for signal magnitude of levels 1 and 0 from the three-dimensional eye diagram, and extract, from the histogram, a statistical count difference between levels 0 and 1 corresponding to on-off keying modulation pattern, and an eye height representing a signal magnitude difference between levels 0 and 1, as the performance parameters proportional to the blood glucose concentration.

The signal receiving module may extract, from the three-dimensional eye diagram, a two-dimensional eye diagram representing actual waveforms for all modulation patterns reconstructed in time, and extract, from the two-dimensional eye diagram, overshoot, jitter, eye width, rise time, fall time, and eye mask as the performance parameters.

The performance parameters extracted from the three-dimensional eye diagram may have a hierarchical structure and may be input as variables in the multivariate analysis performed by a sensing module.

The sensing module may show, based on a result of performing the multivariate analysis, that a statistical count difference and a bit error ratio (BER) between levels 0 and 1 increase as a blood glucose value increases.

The sensing module may show, based on a result of performing the multivariate analysis, that an eye height (eye_height) and a Q_factor between levels 0 and 1 decrease as a blood glucose value increases.

According to another aspect of the present invention, there is provided a method of non-invasive blood glucose monitoring, which includes: transmitting, by a signal transmitting module, a terahertz wireless signal modulated into a PRBS pattern at a terahertz carrier frequency to a body part; demodulating, by a signal receiving module, the terahertz wireless signal reflected from the body part to measure performance parameters of the modulation pattern; and performing, by a sensing module, multivariate analysis using the performance parameters of the modulation pattern measured through the signal receiving module and quantifying features related to blood glucose through the multivariate analysis to measure blood glucose concentration.

In the transmitting of the terahertz wireless signal to the body part, the signal transmitting module may beat two laser optical signals with different wavelengths using a photo mixer to generate a terahertz wireless signal corresponding to a wavelength difference between the two laser optical signals.

The signal transmitting module may project a femtosecond laser into a photoconductor to generate a terahertz signal.

The signal transmitting module may use an electronic-based terahertz transmitter to reduce power consumption.

In the demodulating of the terahertz wireless signal to measure the performance parameters of the modulation pattern, the signal receiving module may remove a carrier frequency from the terahertz wireless signal reflected from the body part through a subharmonic mixer, down-convert the terahertz wireless signal into a signal at a frequency in a baseband, and then reconstruct the modulation pattern to extract performance parameters of distorted modulation pattern proportional to the blood glucose concentration of the body part from a three-dimensional eye diagram.

The signal receiving module may extract a histogram representing a statistical count for signal magnitude from the three-dimensional eye diagram, and extract, from the histogram, a statistical count (probability density function, pdf) difference between levels 0 and 1 corresponding to on-off keying modulation pattern, and an eye height representing a signal magnitude difference between levels 0 and 1, as the performance parameters proportional to the blood glucose concentration.

The signal receiving module may extract, from the three-dimensional eye diagram, a two-dimensional eye diagram representing actual waveforms for all modulation patterns reconstructed in time by overlapping the actual waveforms, and
extract, from the two-dimensional eye diagram, overshoot, jitter, eye width, rise time, fall time, and eye mask as the performance parameters.

The performance parameters extracted from the three-dimensional eye diagram may have a hierarchical structure and may be input as variables in the multivariate analysis performed by a sensing module.

The sensing module may show, based on a result of performing the multivariate analysis, that a statistical count difference and a bit error ratio (BER) between levels 0 and 1 increase as a blood glucose value increases.

The sensing module may show, based on a result of performing the multivariate analysis, that an eye height (eye_height) and a Q_factor between levels 0 and 1 decrease as a blood glucose value increases.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is an exemplary diagram illustrating a schematic configuration of an apparatus for non-invasive blood glucose monitoring according to an embodiment of the present invention;

FIG. 2 is an exemplary diagram illustrating a three-dimensional diagram composed of a histogram showing a statistical count (pdf) of signal magnitude and a two-dimensional eye diagram showing actual waveforms of all modulation patterns reconstructed in time by overlapping the actual waveforms;

FIG. 3A and FIG. 3B are, respectively, an exemplary diagram illustrating performance parameters defined in the two-dimensional eye diagram;

FIG. 4 is an exemplary diagram for describing a hierarchical structure between performance parameters extracted from a three-dimensional eye diagram and a blood glucose monitoring method using a multivariate analysis method;

FIG. 5A, FIG. 5B and FIG. 5C are, respectively, an exemplary diagram illustrating a histogram change graph according to glucose solution concentration;

FIG. 6A and FIG. 6B are, respectively, an exemplary diagram illustrating the performance parameters extracted from the histogram illustrated in FIGS. 5A to 5C according to the glucose solution concentration, including the statistical count difference and the eye height between levels 0 and 1, and the values of the predefined performance parameters (bit error rate and Q_factor);

FIG. 7A and FIG. 7B are, respectively, an exemplary diagram illustrating the results of multivariate analysis utilizing the statistical count difference and the eye height between levels 0 and 1, and the values of the predefined performance parameters (bit error rate, Q_factor) for blood glucose monitoring based on accurate blood glucose prediction; and

FIG. 8 is a flowchart for describing a method of non-invasive blood glucose monitoring according to an embodiment of the present invention.

### DETAILED DESCRIPTION OF EXEMPLARY EMBODIMENTS

Hereinafter, embodiments of an apparatus and method for non-invasive blood glucose monitoring according to the present invention will be described with reference to the attached drawings. In this process, thicknesses of lines, sizes of components, and the like illustrated in the accompanying drawings may be exaggerated for clearness of explanation and convenience. In addition, terms to be described below are defined in consideration of functions in the present disclosure and may be construed in different ways according to the intention of users or practice. Therefore, these terms should be defined on the basis of the content throughout the present specification.

Generally, a terahertz (THz) frequency band (0.1 THz to 10 THz) is a band which allows both wireless communication and sensing functions to be performed.

Conventionally, due to the lack of development of a terahertz (THz) device technology, terahertz applications were limited to sensing and imaging. Recently, technological advancements have been made in terahertz signal generation, modulation, antennas, etc., and it is attracting attention as a band that can be utilized in 6G wireless communication technology.

In particular, as photonics technology and electronic technology are integrated together to develop efficient and programmable devices from the perspective of a system, not only do the wireless transmission speed and transmission capacity increase, but also the convergence between applications such as sensing and imaging using terahertz waves has enabled high resolution, leading to the emergence of new application services.

Accordingly, the present invention provides a method of increasing resolution and accuracy of a method of non-invasive blood glucose measurement by analyzing performance parameters of data for terahertz reflection signals using a multivariate analysis method to extract characteristics that are highly correlated with blood glucose concentration and also considering the relationship between variables.

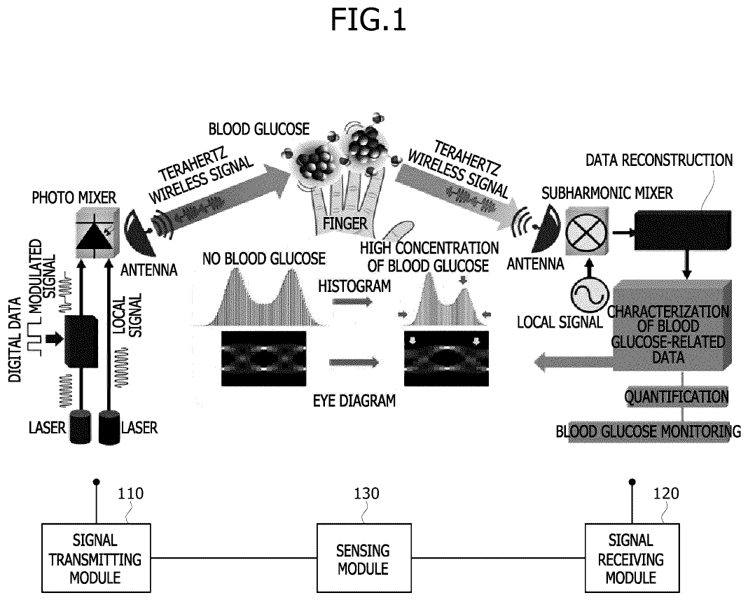

FIG. 1 is an exemplary diagram illustrating a schematic configuration of an apparatus for non-invasive blood glucose monitoring according to an embodiment of the present invention.

Referring to FIG. 1, the apparatus for non-invasive blood glucose monitoring according to the present embodiment includes a signal transmitting module 110, a signal receiving module 120, and a sensing module 130.

In this case, the sensing module 130 may include a processor or may be replaced with the processor to perform the corresponding function.

The signal transmitting module 110 transmits a terahertz wireless signal modulated into a PRBS pattern at a terahertz carrier frequency to a body part (e.g., a finger).

Here, two laser optical signals with different wavelengths are beaten using a photo mixer to generate a terahertz wireless signal corresponding to the wavelength difference between the two laser optical signals.

This method has the advantage of being able to easily change a frequency of the terahertz wireless signal depending on materials to be detected because a resonance frequency is different depending on the materials.

In addition, the problem of artificial noise between laser signals with two wavelengths may be solved by projecting a femtosecond laser into a photoconductor to generate terahertz.

In addition, an electronic-based terahertz transmitter may be used to reduce power consumption at a system level.

Accordingly, the terahertz wireless signal passes through skin tissue of a finger and causes bound water molecules attached into blood glucose molecules (hydration water) in blood vessels to vibrate and reorient, and some energy is absorbed, thereby distorting the terahertz reflection signal (i.e., the terahertz wireless signal reflected from the finger).

In this case, a modulation rate is very closely related to the dynamics in which a hydrogen bond between the glucose molecules and water molecules in the blood vessels vibrates.

Therefore, the sensing module 130 modulates signal having a modulation rate of a time scale similar to the dynamics of a vibration mode between molecules into a wireless signal of a terahertz carrier frequency, outputs the wireless signal to a part where blood glucose is to be measured, and extracts characteristics proportional to the blood glucose concentration among the performance degradation of the modulation pattern of the reflected terahertz wireless signal, thereby accurately monitoring the blood glucose in a non-invasive manner.

The signal receiving module 120 demodulates the terahertz wireless signal reflected from the body part (e.g., the finger) to measure the performance parameters of the modulation pattern.

The signal receiving module 120 removes a carrier frequency of the terahertz wireless signal reflected from the finger through a subharmonic mixer (i.e., subharmonic mixer), frequency-down converts the terahertz wireless signal into a signal at a frequency in a baseband, and then reconstructs modulation pattern, thereby extracting performance parameters of distorted modulation pattern in proportion to the blood glucose concentration in the finger.

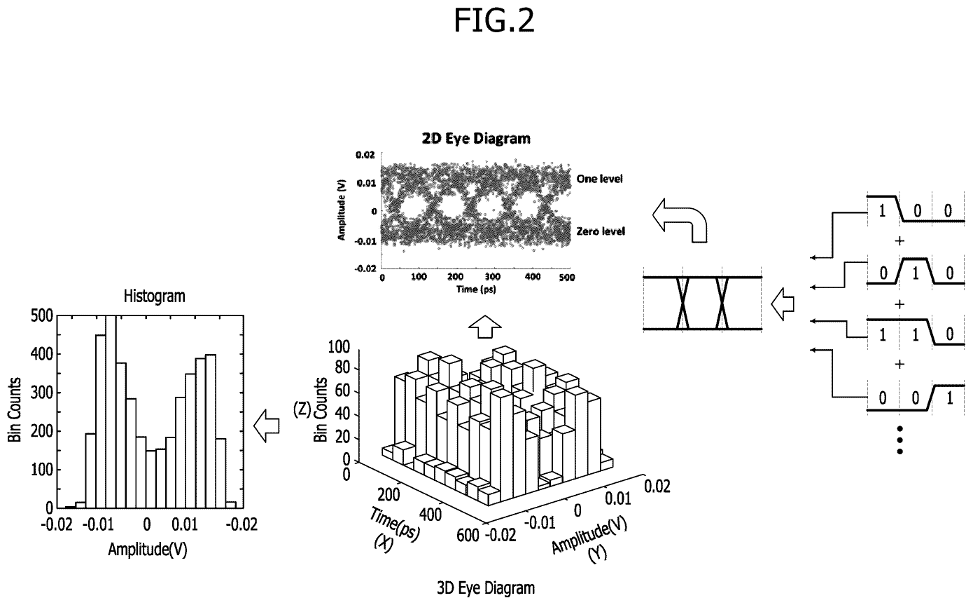

For example, as illustrated in FIG. 2, a histogram representing a statistical count (probability) for signal magnitude is extracted from a three-dimensional (3D) eye diagram.

FIG. 2 is an exemplary diagram illustrating a three-dimensional diagram composed of a histogram showing a statistical count (pdf) for signal magnitude and a two-dimensional eye diagram showing actual waveforms of all modulation patterns reconstructed in time by overlapping the actual waveforms.

The sensing module 130 performs multivariate analysis using the performance parameters measured through the signal receiving module 120 and precisely measures the blood glucose concentration by quantifying blood glucose-related features in a complex and multifaceted manner through this multivariate analysis.

In this case, a statistical count difference between levels 0 and 1 corresponding to on-off keying modulation pattern, and an eye height representing a signal magnitude difference between levels 0 and 1 may be extracted from a histogram as the performance parameters proportional to the blood glucose concentration.

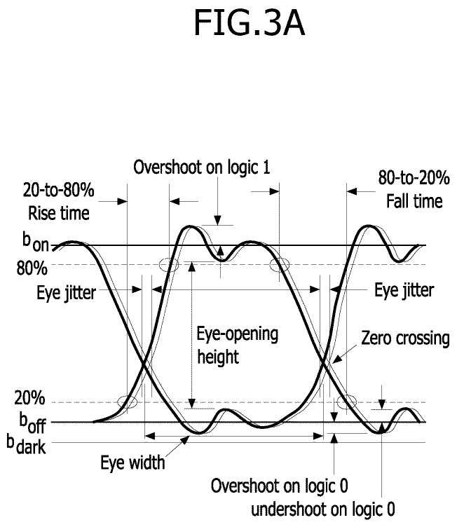

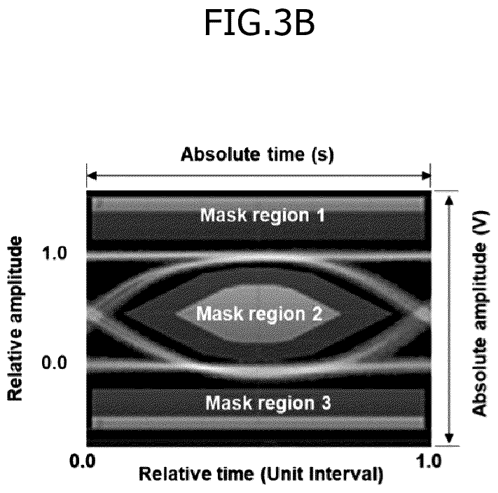

In addition, in a two-dimensional (2D) eye diagram that represents actual waveforms for modulation pattern reconstructed in time, more specific performance parameters (e.g., overshoot, jitter, eye width, rise time, fall time, eye mask, etc.) of the modulation signal may be extracted, as illustrated in FIG. 3.

FIG. 3 is an exemplary diagram illustrating the performance parameters defined in the 2D eye diagram. Specifically, FIG. 3A is an exemplary diagram illustrating the performance parameters of the overshoot, jitter, eye width, rise time, fall time, and FIG. 3B is an exemplary diagram illustrating performance parameters of eye mask.

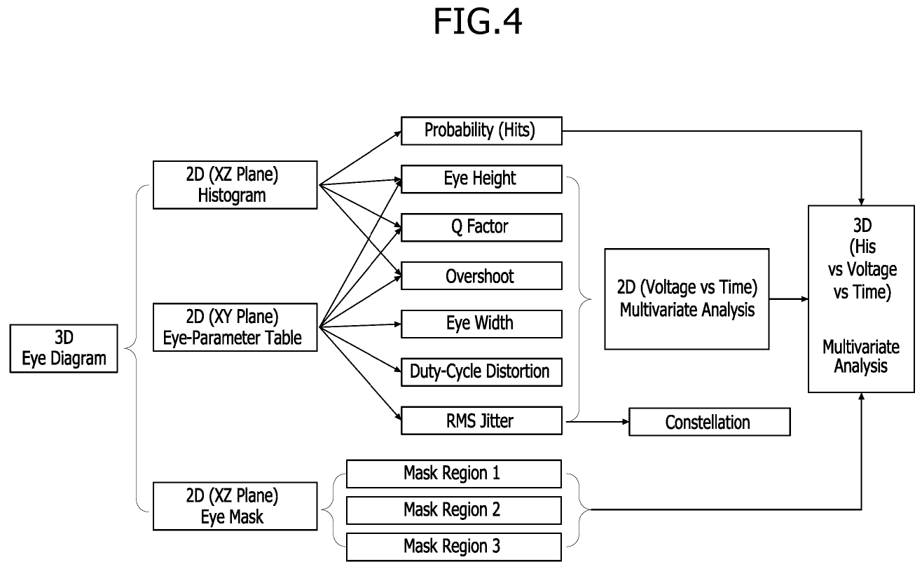

In addition, the performance parameters extracted from the 3D eye diagram have a hierarchical structure as illustrated in FIG. 4 and are input as variables in multivariate analysis.

FIG. 4 is an exemplary diagram for describing a hierarchical structure between performance parameters extracted from a 3D eye diagram and a blood glucose monitoring method using a multivariate analysis method.

In the multivariate analysis, the performance parameters are not analyzed individually but are analyzed all at once to consider the relationship between the variables.

In this case, the 3D eye diagram includes a 2D histogram, a 2D eye-parameter table, and a 2D eye mask.

Specifically, the 2D histogram includes probability, eye height, Q_factor, and overshoot, the 2D eye-parameter table includes eye width, duty-cycle distortion, and RMS jitter, and the 2D eye mask includes mask region 1, mask region 2, and mask region 3.

In addition, as the multivariate analysis method, principle component analysis, regression, and multivariate adaptive regression splines (MARS) may be applied.

In this embodiment, the correlation between the blood glucose concentration and the terahertz performance parameters is increased to improve accuracy.

For reference, the performance parameters used as the variables in the multivariate analysis may be defined by the formulas shown in Table 1.

Table 1 shows definitions of the terahertz performance parameters used as the variables in the multivariate analysis for the blood glucose monitoring in this embodiment.

TABLE 1

| Eye Parameter | Definition |
|---|---|
| Eye height | (Vₜₒₚ −3σₜₒₚ) − (V_{base} +3σ_{base}) |
| Eye width | (t_{crossing2} − 3σ_{crossing}) − (t_{crossing1} + 3σ_{crossing}) |
| RMS jitter | σ_{crossing} |
| Q factor | $\frac{{V}_{top} - {V}_{base}}{{σ}_{top} + {σ}_{base}}$ |
| Overshoot | $\frac{{V}_{top} + {V}_{95}}{{V}_{top} - {V}_{base}} \times 100 \%$ |
| Duty-cycle distortion | $\frac{\vert {t}_{r 50 \%} - {t}_{f 50 \%} \vert}{{t}_{crossing 2} + {t}_{crossing 1}} \times 100 \%$ |

Here, Vₜₒₚ refers to a mean magnitude of logic level 1, V_{base} refers to a mean magnitude of logic level 0, σ_{crossing} refers to a standard deviation for the time when logic levels 0 and 1 intersect, σₜₒₚ refers to a standard deviation of a statistical count for logic level 1, σ_{base} refers to a standard deviation of a statistical count for logic level 0, t_{r50%} refers to the time for 50% amplitude level of a rising modulation pattern edge, t_{r50%} refers to the time for 50% amplitude level of a falling modulation pattern edge, t_{crossing2} refers to the time for second crossing of logic levels 0 and 1, t_{crossing1} refers to the time for first crossing of logic levels 0 and 1, and V95 refers to 95% of amplitude distribution extending upward from mean logic level 1 to a maximum value.

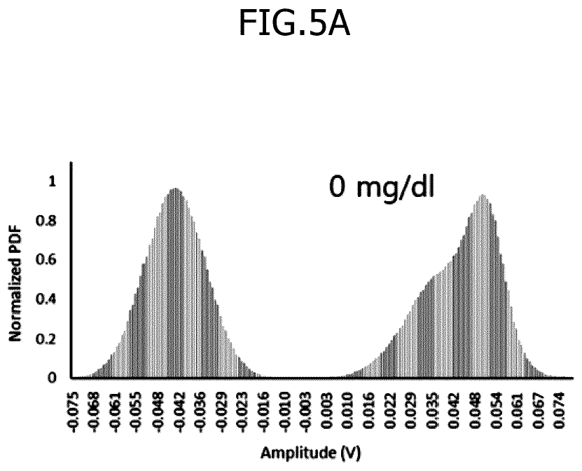

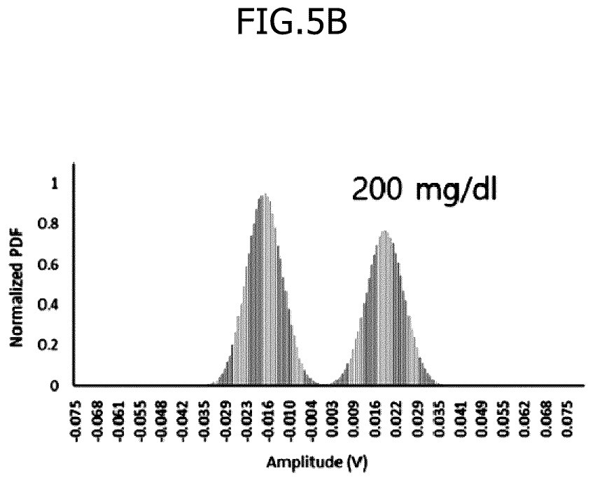

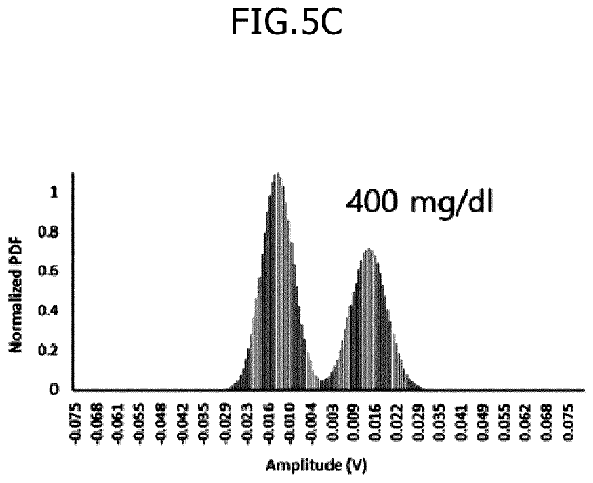

FIG. 5 is an exemplary diagram illustrating a histogram change graph according to glucose solution concentration. FIG. 5A is a histogram change graph when the glucose solution concentration is 0 mg/dl, FIG. 5B is a histogram change graph when the glucose solution concentration is 200 mg/dl, and FIG. 5C is a histogram change graph when the glucose solution concentration is 400 mg/dl.

FIG. 5 is a histogram that clearly illustrates the changes in the performance parameters according to the glucose solution concentration, including the change in the statistical count (probability) difference between levels 0 and 1 and the change in the eye height representing the signal magnitude difference between levels 0 and 1.

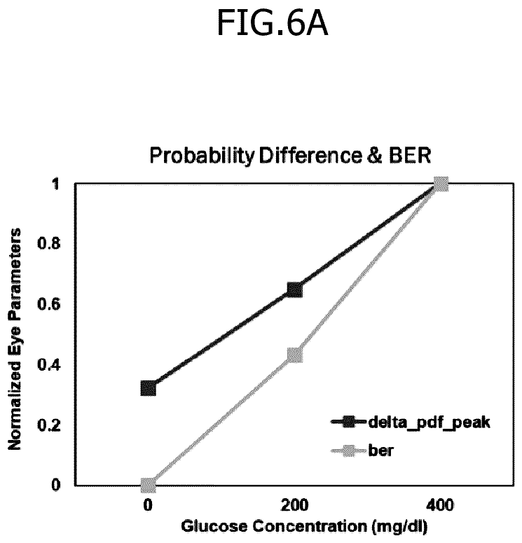

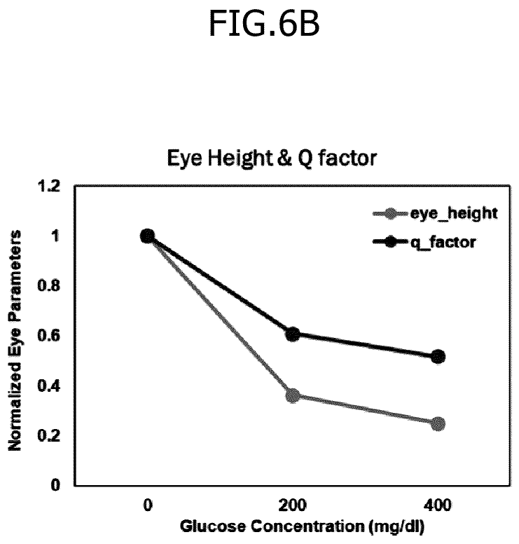

FIG. 6 is an exemplary diagram illustrating the performance parameters extracted from the histogram illustrated in FIG. 5 according to the glucose solution concentration, including the statistical count difference and the eye height between levels 0 and 1, and the values of the performance parameters (bit error rate and Q_factor) predefined in Table 1.

Referring to FIG. 6, it can be seen that the statistical count difference and the bit error ratio increase as the glucose solution concentration increases (i.e., as the blood glucose value increases), while the eye height (eye_height) and the Q_factor decrease as the glucose solution concentration increases (i.e., as the blood glucose value increases).

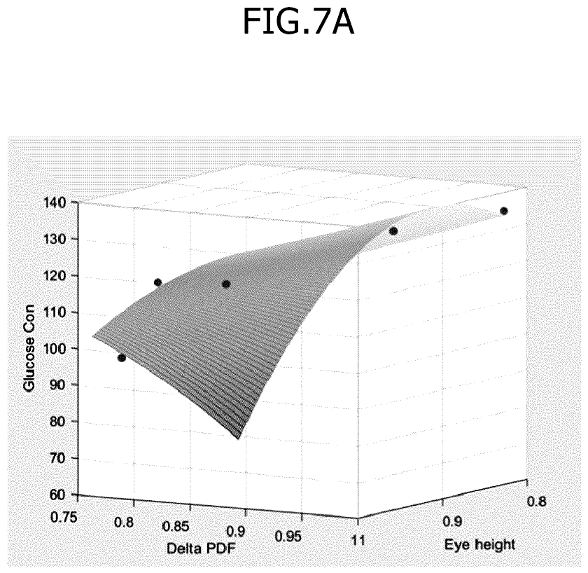

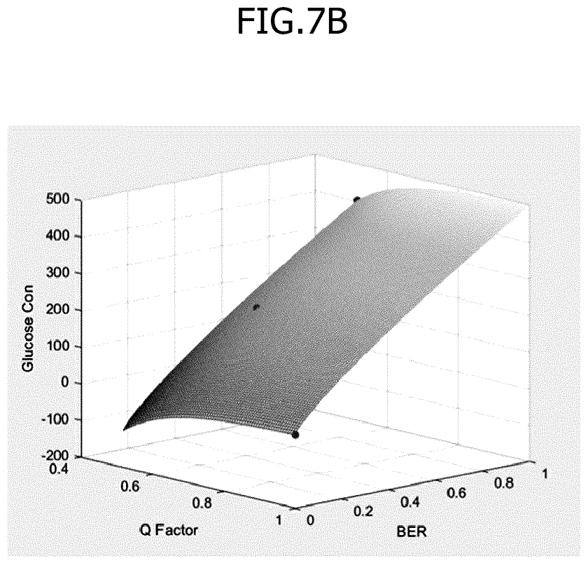

FIG. 7 is an exemplary diagram illustrating the results of the multivariate analysis utilizing the statistical count difference, the eye height, and the values of the predefined performance parameters (bit error rate, Q_factor) for the blood glucose monitoring based on the accurate blood glucose prediction.

As illustrated in FIG. 7A, a correlation equation for the blood glucose concentration (glucose solution concentration) for the statistical count difference and the eye height is as shown in the following Equation 1, and a correlation coefficient is 98.2%.

$${C}_{g} = 347.2 - 1.62 \times [ - 715.4 + 1761 \times {Δ}_{PDF} - 912.2 \times {Δ}_{PDF}^{2} ] - 3.29 \times [ 806 \times {e}^{- 2.1 \times {H}_{eye}} ] + 0.0249 \times [ - 715.4 + 1761 \times {Δ}_{PDF} - 912.2 \times {Δ}_{PDF}^{2} ] \times [ 806 \times {e}^{- 2.1 \times {H}_{eye}} ] \qquad [ Equation 1 ]$$

C_{g}: glucose solution concentration, Δ_{PDF}: statistical count difference, H_{cuc}: eye height

In addition, as illustrated in FIG. 7B, the correlation equation of the blood glucose concentration (glucose solution concentration) for the bit error rate and the Q_factor is as shown in the following Equation 2, and the correlation coefficient is 100%.

$${C}_{g} = - 0.25 \times [ 2.095 \times {10}^{4} \times {e}^{- 7.65 \times Q} ] + 1.25 \times [ 400 \times {BER}^{0.82} ] \qquad [ Equation 2 ]$$

C_{g}: glucose solution concentration, Q: statistical count difference, BER: bit error rate

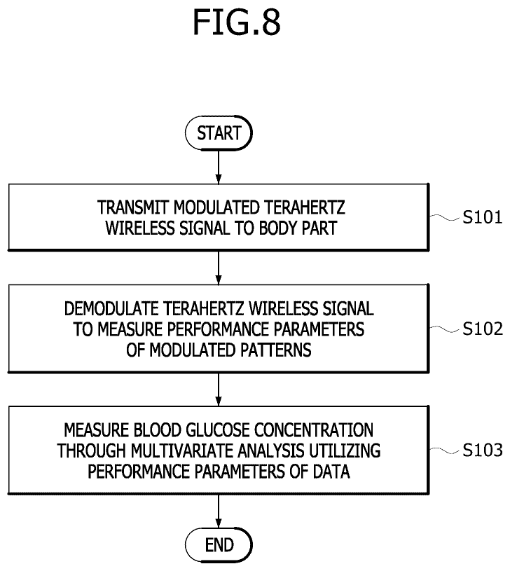

FIG. 8 is a flowchart for describing a method of non-invasive blood glucose monitoring according to an embodiment of the present invention.

As illustrated in FIG. 8, the sensing module 130 transmits the terahertz wireless signal, in which signals are modulated at the terahertz carrier frequency, to a body part (e.g., a finger) through the signal transmitting module 110 (S101).

The sensing module 130 demodulates the terahertz wireless signal reflected from the body part (e.g., the finger) through the signal receiving module 120 and measures the performance parameters of the modulation pattern (S102).

The sensing module 130 performs the multivariate analysis by utilizing the performance parameters of the modulation pattern measured through the signal receiving module 120, thereby quantifying the blood glucose-related features and measuring the blood glucose concentration (S103).

The signal transmitting module 110 generates the terahertz wireless signal corresponding to the wavelength difference between two laser optical signals by beating the two laser optical signals with different wavelengths using a photo mixer, and generates a terahertz signal by projecting a femtosecond laser into a photoconductor.

The signal receiving module 120 removes the carrier frequency from the terahertz wireless signal reflected from the body part through the subharmonic mixer, down-converts the terahertz wireless signal into a signal at a frequency in the baseband, and then reconstructs the modulation pattern, thereby extracting the performance parameters of the distorted modulation pattern proportional to the blood glucose concentration of the body part from the 3D eye diagram.

The 3D eye diagram includes a histogram representing the statistical count for the signal magnitude and the 2D eye diagram representing actual waveforms for all modulation patterns of reconstructed in time by overlapping the actual waveforms.

In this case, the histogram includes, as the performance parameters proportional to the blood glucose concentration, the statistical count difference between levels 0 and 1 corresponding to on-off keying modulation pattern and the eye height representing the signal magnitude difference between levels 0 and 1, and the 2D eye diagram includes, as the performance parameters, overshoot, jitter, eye width, rise time, fall time, and eye mask.

The sensing module 130 uses the performance parameters extracted from the 3D eye diagram as the variables in the multivariate analysis. Accordingly, according to the results of performing the multivariate analysis, it can be seen that the statistical count difference and the bit error ratio between levels 0 and 1 increase as the blood glucose value increase, while the eye height and the Q_factor decrease as the blood glucose value increases.

As described above, the present embodiment precisely measures the blood glucose concentration by quantifying the blood glucose-related features in a complex and multifaceted manner through the multivariate analysis by utilizing the performance parameters of the reflected terahertz wireless signal after projecting the pattern modulated at the terahertz carrier frequency into the finger.

In addition, according to the present embodiment, the terahertz wireless signal that causes bound water molecules attached into blood glucose molecules (hydration water) in blood vessels to vibrate and reorient may be used, and using the modulation rate that has a very direct effect on the dynamics in which the hydrogen bonds between glucose molecules and water molecules in the blood vessels vibrate, it is possible to effectively extract characteristics proportional to the blood glucose concentration.

In addition, according to the present embodiment, by considering the relationship between the variables through the multivariate analysis of the performance parameters of distorted modulation pattern proportional to the blood glucose concentration in the finger, such as the statistical count difference between levels 0 and 1, the eye height indicating the signal magnitude difference between levels 0 and 1, and the performance parameters (e.g., overshoot, jitter, eye width, rise time, fall time, eye mask, etc.), the correlation between the blood glucose concentration and the terahertz performance parameters increases compared to individual analysis, thereby improving the accuracy of the non-invasive blood glucose monitoring.

In addition, according to the present embodiment, by combining the advantages of the wireless communication technology and the photonics technology by the method of non-invasive blood glucose monitoring that measures the blood glucose concentration using the terahertz wireless communication technology, it is possible to overcome the technical limitations of the existing method of non-invasive blood glucose monitoring, and furthermore, by analyzing the performance parameters highly correlated with the blood glucose concentration in the three-dimensional and multifaceted manner using the multivariate analysis method, it is possible to overcome the limitations of the existing blood glucose monitoring technology.

According to the apparatus and method for non-invasive blood glucose monitoring of the present invention, it is possible to improve the resolution and accuracy of blood glucose measurement through the non-invasive monitoring using the terahertz wireless communication technology and compensate for the individual differences in the signal magnitude without requiring additional devices or signal processing.

Although the present invention has been described with reference to embodiments shown in the accompanying drawings, these are only examples. It will be understood by those skilled in the art that various modifications and other equivalent exemplary embodiments are possible from the present invention. Accordingly, the true technical scope of the present invention is to be determined from the spirit of the appended claims. Implementations described herein may be implemented in, for example, a method or process, an apparatus, a software program, a data stream, or a signal. Even when discussed only in the context of a single form of implementation (e.g., discussed only as a method), implementations of the discussed features may also be implemented in other forms (e.g., an apparatus or a program). The apparatus may be implemented in suitable hardware, software, firmware, and the like. A method may be implemented in an apparatus such as a processor, which generally refers to a computer, a microprocessor, an integrated circuit, a processing device including a programmable logic device, or the like.

What is claimed is:

1. An apparatus for non-invasive blood glucose monitoring, comprising:
a signal transmitting module that transmits a terahertz wireless signal modulated into a PRBS pattern at a terahertz carrier frequency to a body part;
a signal receiving module that demodulates the terahertz wireless signal reflected from the body part to measure performance parameters of the modulation pattern; and
a sensing module that performs multivariate analysis using the performance parameters of the modulation pattern measured through the signal receiving module and quantifies features related to blood glucose through the multivariate analysis to measure blood glucose concentration.

2. The apparatus of claim 1, wherein the signal transmitting module beats two laser optical signals with different wavelengths using a photo mixer to generate a terahertz wireless signal corresponding to a wavelength difference between the two laser optical signals.

3. The apparatus of claim 2, wherein the signal transmitting module projects a femtosecond laser into a photoconductor to generate a terahertz signal.

4. The apparatus of claim 2, wherein the signal transmitting module uses an electronic-based terahertz transmitter to reduce power consumption.

5. The apparatus of claim 1, wherein the signal receiving module removes a carrier frequency from the terahertz wireless signal reflected from the body part through a subharmonic mixer, down-converts the terahertz wireless signal into a signal at a frequency in a baseband and then reconstructs the modulation pattern to extract performance parameters of distorted modulation pattern proportional to the blood glucose concentration of the body part from a three-dimensional eye diagram.

6. The apparatus of claim 5, wherein the signal receiving module extracts a histogram representing a statistical count for signal magnitude of levels 1 and 0 from the three-dimensional eye diagram, and
extracts, from the histogram, a statistical count difference between levels 0 and 1 corresponding to on-off keying modulation pattern, and an eye height representing a signal magnitude difference between levels 0 and 1, as the performance parameters proportional to the blood glucose concentration.

7. The apparatus of claim 5, wherein the signal receiving module extracts, from the three-dimensional eye diagram, a two-dimensional eye diagram representing actual waveforms for all modulation patterns reconstructed in time, and
extracts, from the two-dimensional eye diagram, overshoot, jitter, eye width, rise time, fall time, and eye mask as the performance parameters.

8. The apparatus of claim 5, wherein the performance parameters extracted from the three-dimensional eye diagram have a hierarchical structure and are input as variables in the multivariate analysis performed by a sensing module.

9. The apparatus of claim 1, wherein the sensing module shows, based on a result of performing the multivariate analysis, that a statistical count difference and a bit error ratio (BER) between levels 0 and 1 increase as a blood glucose value increases.

10. The apparatus of claim 1, wherein the sensing module shows, based on a result of performing the multivariate analysis, that an eye height (eye_height) and a Q_factor between levels 0 and 1 decrease as a blood glucose value increases.

11. A method of non-invasive blood glucose monitoring, comprising:
transmitting, by a signal transmitting module, a terahertz wireless signal modulated into a PRBS pattern at a terahertz carrier frequency to a body part;
demodulating, by a signal receiving module, the terahertz wireless signal reflected from the body part to measure performance parameters of the modulation pattern; and
performing, by a sensing module, multivariate analysis using the performance parameters of the modulation pattern measured through the signal receiving module and quantifying features related to blood glucose through the multivariate analysis to measure blood glucose concentration.

12. The method of claim 11, wherein, in the transmitting of the terahertz wireless signal to the body part, the signal transmitting module beats two laser optical signals with different wavelengths using a photo mixer to generate a terahertz wireless signal corresponding to a wavelength difference between the two laser optical signals.

13. The method of claim 12, wherein the signal transmitting module projects a femtosecond laser into a photoconductor to generate a terahertz signal.

14. The method of claim 12, wherein the signal transmitting module uses an electronic-based terahertz transmitter to reduce power consumption.

15. The method of claim 11, wherein, in the demodulating of the terahertz wireless signal to measure the performance parameters of the modulation pattern, the signal receiving module removes a carrier frequency from the terahertz wireless signal reflected from the body part through a subharmonic mixer, down-converts the terahertz wireless signal into a signal at a frequency in a baseband, and then reconstructs the modulation pattern to extract performance parameters of distorted modulation pattern proportional to the blood glucose concentration of the body part from a three-dimensional eye diagram.

16. The method of claim 15, wherein the signal receiving module extracts a histogram representing a statistical count for signal magnitude from the three-dimensional eye diagram, and
extracts, from the histogram, a statistical count difference between levels 0 and 1 corresponding to on-off keying modulation pattern, and an eye height representing a signal magnitude difference between levels 0 and 1, as the performance parameters proportional to the blood glucose concentration.

17. The method of claim 15, wherein the signal receiving module extracts, from the three-dimensional eye diagram, a two-dimensional eye diagram representing actual waveforms for all modulation patterns reconstructed in time by overlapping the actual waveforms, and
extracts, from the two-dimensional eye diagram, overshoot, jitter, eye width, rise time, fall time, and eye mask as the performance parameters.

18. The method of claim 15, wherein the performance parameters extracted from the three-dimensional eye diagram have a hierarchical structure and are input as variables in the multivariate analysis performed by a sensing module.

19. The method of claim 11, wherein the sensing module shows, based on a result of performing the multivariate analysis, that a statistical count difference and a bit error ratio (BER) between levels 0 and 1 increase as a blood glucose value increases.

20. The method of claim 11, wherein the sensing module shows, based on a result of performing the multivariate analysis, that an eye height (eye_height) and a Q_factor between levels 0 and 1 decrease as a blood glucose value increases.
